(19) United States
(12) Patent Application Publication
(10) Pub. No.: US 2002/0106685 A1
(43) Pub. Date: Aug. 8, 2002

(54) Method for specifically detecting tumor cells and their precursors in uterine cervical smears by simultaneously measuring at least 2 different molecular markers

(76) Inventors: Guido Henning, Koln (DE); Ralph Wirtz, Koln (DE); Margit Doth, Leverkusen (DE); Kerstin Bohmann, Krefeld (DE); Sylvia Unger, Heidelberg (DE)

Correspondence Address: Jeffrey M. Greenman, Vice President, Patents and Licensing, Bayer Corporation, 400 Morgan Lane, West Haven, CT, 06516, US

(21) Appl. No.: 10/022,618

(22) Filed: Dec. 17, 2001

(30) Foreign Application Priority Data
Dec. 18, 2000 (DE) 10063179.7

(51) Int. Cl. C12Q 1/68; G01N 33/574

(52) U.S. Cl. 435/6; 435/7.23

(57) ABSTRACT

The present invention relates to an automatable method for obtaining an improved diagnosis of cancer, and its precancerous stages in uterine cervical smears by simultaneously staining and detecting at least two different molecular markers, which exhibit a disease-associated change in gene expression, in a cell by means of using antibodies or nucleic acid probes.

### STATE OF THE ART

Cancer diseases are still one of the most frequent causes of death worldwide. There is a clinical requirement for the early recognition and specific detection of cancer and pre-cancerous stages in order to prevent tumour development by early therapeutic intervention. For this reason, prevention programmes for various carcinomas (in particular cervix, breast and intestine) have already been on offer since the 1950s, with these programmes having led to declining mortality rates in many countries.

In the case of cervical cancer in women, the medical check-up is essentially based on a morphological/cytological examination of cell smears taken from the cervix, i.e. what is termed the PAP test. However, this test is of only limited sensitivity (up to 40% falsely negative diagnoses, Duggan et al.; 1998, Eur J Gynaecol Oncol; 19:209-214; Bishop et al., 1996, Buss Pan Am Health Organ, 30, 378-86). Furthermore, up to 10% of the smears are classified as ASCUS (atypical squamous cells of undetermined significance), i.e. it is not possible to make a clear categorization into normal, moderate or severe lesion or tumour. However, experience shows that up to 10% of this ASCUS population consist of true lesions, which are consequently also overlooked (Manos et al. 1999, JAMA 281, 1605-1610). If abnormal cells are discovered, additional diagnostic procedures, such as colposcopy or the removal of a biopsy, are carried out, with it being possible to use the results of these procedures to reach a more accurate diagnosis and to take a decision on therapy, e.g. surgical removal of the diseased region or laser therapy.

A large number of genes which, as a consequence of mutations, give rise to a change in the expression of their proteins, and which consequently play a role in the development of cancer, have been identified in recent years. The corresponding proteins can potentially be detected, as diagnostic markers, both in the serum and at a cellular level. The proteins are involved in physiological regulation processes in the cells, such as growth control (proliferation genes, e.g. Ki67 and mcm-5; oncogenes and tumour suppressor genes, e.g. p16, EGF receptor, her2/neu and mdm-2), apoptosis (natural cell death, e.g. p53 and bcl-2), DNA repair (msh-1) and cell adhesion (E-cadherin, β-catenin and APC). Examples of suitable methods for detecting the markers are immunohistochemical and immunocytochemical detection methods which use specific antibodies against these proteins (van Noorden, 1986, Immunocytochemistry, Modern Methods and Applications, 2nd edition, Wright, Bristol, 26-53).

In addition, in certain types of cancer, the appearance of viruses is significantly associated with development of the cancer. Viral DNA is detected by hybridization methods and also serves as a diagnostic marker e.g. when detecting human papillomaviruses in cervical smears (detection kits can be obtained from several suppliers, e.g. Digene, DAKO, BioGenex and ENZO).

It has been shown on many occasions that a single marker is not sufficiently specific for recognizing pathologically altered cells since it is also partly present in healthy cells. This is based on the fact that markers are proteins which are involved in physiological regulation processes in healthy cells as well. Thus, while HPV, for example, can be detected in virtually all cervical tumours, it can also be detected in many normal cervical epithelia; i.e. while a test for detecting HPV is highly sensitive, it is only of low specificity (e.g. Cuzick, 2000, JAMA, 283, 108-109). Williams et al. (1998, PNAS, 95, 14932-14937) describe the DNA replication marker mcm-5, which detects 28 tested lesions/tumours of the cervix (n=58) but also gives a falsely positive staining of 2 out of 28 normal smears. Sano et al. (Pathology Intl., 1998, 48, 580-585) report that the marker p16 nonspecifically stains 3 out of 15 normal cervical biopsies.

In cytology/pathology, there is increasing interest in detecting several markers in a biological sample. On the one hand, this makes more information available; on the other hand, nonspecific stainings obtained with individual markers can be more easily recognized for what they are. Traditionally, serial sections of an available biopsy or repeated preparations of a body sample (e.g. cervical smear in fixative) are prepared for this purpose, with each then being stained with a particular marker. This method suffers from several disadvantages: the fact that it consumes a large amount of the patient sample material, which is usually limited, and of staining reagents, the fact that it takes a great deal of time and is expensive, and also the fact that there is no possibility of unambiguously colocating two or more markers in a cell. While the simultaneous detection of two or more different biological markers in a biological sample has already been described in the literature (DAKO, Practical Guide for Immunoenzymatic Double Staining Methods; Gomez et al., Eur. J. Histochem., 1997 41. 255-259), this approach has not so far been used in routine diagnosis. Terhavauta et al. (Cytopathology, 1994, 5, 282-293) use a double staining of p53 and Ki67 on biopsies taken from cervical lesions and can detect both markers in basal and parabasal cells derived from HPV-positive and HPV-negative lesions, in some cases in one cell. The above-described double staining was carried out on cervical biopsies and cannot be transferred to cervical smears. In the same way, the cell composition of a smear, which contains scarcely any basal and parabasal cells, is different from that of a biopsy. According to our findings, it is not possible to obtain any diagnostically utilizable result when applying the above-described marker combination to cervical smears. In addition, the authors do not use the information obtained from the double staining to reach, by combining the results, a more accurate conclusion as regards the presence of tumour cells in this sample. et al. (1998, Cancer Epidemiology, Biomarkers and Evolution, 7, 1027-1033) describe the multiple staining of different tumour-associated markers (DNA content, G actin and p53) in needle puncture material (FNA, fine needle aspirates) obtained from breast tissue. They interpret their results to the effect that using several markers leads to a higher clinical specificity than does observation of the individual markers and can consequently play an important role in the early recognition of breast cancer. The multiple staining cited by the authors is restricted to needle puncture material obtained from breast tissue. There is no attempt to use the staining information with a view to automating the detection of tumour regions. In addition to this, the staining methodology which is described is not applicable to other biological materials (i.e. biopsies and cervical smears) because of the different properties of the substances.

### Automation

A further improvement in current clinical tumour diagnosis consists both in the automation of sample preparation and staining and in automated image analysis including diagnosis establishment. In addition to the savings in time and personnel, this also results in more objective, and consequently more uniform diagnosis. The staining of biological markers in tumour cells, or their precursors, using fluorescence or chromogenic dyes permits quantification, and consequently automated readout, of the signals. There already exist systems which permit automated implementation of staining protocols, although without automated diagnosis by means of image analysis (LeNeel et al., 1998, Clin Chim Acta, 278, 185-192). The system which is most advanced is the automated detection of tumour cells and their precursors in cervical smears using morphological image information (Sawaya et al., 1999, Clinical Obstetrics and Gynaecology, 42, 922-928; Stoler, 2000 Mod. Pathol., 13, 275-284).

In addition to the automated morphological detection of abnormal cells in cervical smears using the PAPNET system, Boon et al. (1995, Diagn. Cytopathol., 13, 423-428) also make use of the immunohistochemical staining of proliferating cells with Ki67 antibodies. However, the use of this one molecular marker does not make it possible to distinguish benign proliferating cells and carcinogenic cells unambiguously.

The patent authored by Boon et al. (U.S. Pat. No. 5,544,650) reports that staining with an immunochemical marker facilitates the automated detection of proliferating (carcinogenic and non-carcinogenic) cells in a sample and that, in a semiautomated process, the monitoring pathologist/cytologist then decides whether the stained cells actually are carcinogenic cells.

The patent U.S. Pat. No. 6,005,256, whose authors are McGlynn and Akkapeddi, describes in detail an appliance and a method for simultaneously detecting several fluorescence-labelled markers in a body sample, also for identifying cancer cells, without, however, going into any specific application in cancer diagnosis or specifying appropriate marker combinations giving increased specificity.

It is known that Ampersand Medical Systems Group (www.ampersandmedical.com) is developing a new screening system for cervical smears (InPath) which, in addition to the company's in-house sample preparation, also involves the fluorescence detection of unspecified biological markers.

### OBJECT

The present invention comprises methods which can be used to diagnose carcinoma cells, or their precursors, at an early stage, and more reliably than previously, in prepared cervical smears.

According to the invention, this is achieved by means of the subject-matter contained in the patent claims.

### SUMMARY OF THE DESCRIPTION/OBJECT ACHIEVEMENT

The present invention is based on the applicants' findings that the simultaneous detection of at least two molecular markers, namely disease-associated changes in gene expression or viral nucleic acids, increases the specificity of the detection of pathologically altered cells, e.g. carcinoma cells and their precursors, in cervical smears and, because of the informative value of combined marker stainings, makes possible a detection which is more specific, and, in addition to this, can be automated as well.

### DETAILED DESCRIPTION

The invention relates to molecular markers which, on being detected individually, do not achieve sufficient specificity with regard to recognizing pathologically altered cells or tissues since they are also partly present, in similar or different quantities, in biological material which is not pathologically altered. This is based on the fact that these markers can be proteins which are involved in physiological regulation processes in healthy cells as well. In addition to this, because of the antibody crossreacting nonspecifically, the detection of the molecular marker may not be unambiguous, with this being manifested in the staining of particles of the biological material which do not contain the molecular marker. The inventors have made the observation that the deficient specificity associated with the detection of single markers can be offset, so as to ensure higher specificity when detecting abnormal cells or tissue sections, by simultaneously detecting at least two markers in a cell. Consequently, greater informative value in the diagnosis of biological samples is achieved by combining several markers in a cell than by using single markers.

The marker combinations involve the visualization of the expression of genes belonging to at least one of the following gene classes: oncogenes, tumour suppressor genes, apoptosis genes, proliferation genes, repair genes and viral genes, or the visualization of an altered expression of genes from at least one of the listed gene classes in combination with the visualization of viral nucleic acids. Preferred combinations contain the following molecular markers: her2/neu, p16, p53, MN, mdm-2, bcl-2 and EGF receptor and DNA from the HPV subtypes 6, 11, 16. 18, 30, 31, 33, 35, 45, 51 and 52. The following combinations are particularly preferred: her2/neu with p16 or EGF-R with p16 or p53 with her2/neu or her2/neu with mdm-2 or bcl-2 with p16 or bcl-2 with her2/neu or p16 with p53.

According to the invention, the applicants' findings are used for a method for the early diagnosis of disease-associated cells or tissue sections which comprises detecting combinations of the described molecular markers with the aim of using automation to detect carcinomas and their precursors. The automated and specific detection of tumour cells can be ensured by the following method: firstly, at least two signals must be present in a cell and, secondly, the signal for each of the two markers must in each case be greater than an individually defined intensity or an individually defined threshold value. By using these two criteria, it is possible to regard those cells which, for example, express a marker above the set threshold, or exhibit stainings for the two markers which are below the respectively defined signal strength, as being healthy.

The expression “molecular marker” comprises molecular changes in cells, in particular changes in gene expression, which have been observed in connection with a cell constitution which is altered or is pathological. Methods for detecting molecular markers comprise any methods which determine the quantity or the presence of the markers, either at the nucleic acid level or the protein level. For detection at the protein level, it is possible to use antibodies or other specific binding proteins (e.g. anti-cullins) which permit subsequent cytochemical or histochemical identification using chromogenic and/or fluorescent detection. For detection at the nucleic acid level, it is possible to use hybridization technologies which, sometimes after additional amplification steps as well (e.g. immunocytochemical amplification of labelled probes after binding to the target sequences), can likewise be identified using cytochemical or histochemical staining reactions.

The expression “simultaneous detection of at least two molecular markers” encompasses methods which visualize the expression of at least two genes in a body sample, in particular in a single preparation of the body sample as well, preferably in a single cell, such that the gene expressions can be observed in connection with each other.

The expression “informative value of combined marker stainings” encompasses the combination of at least two amounts of information which have been obtained on the basis of detecting at least two markers in a body sample, preferably in a single cell. In addition, healthy cells can be distinguished specifically from diseased cells by defining threshold values for the marker intensities.

The expression “pathologically altered cervical smear cells” encompasses carcinomas and their precursors which are derived from the uterovaginal tract of women and which, by routine gynaecological sampling, are applied to a microscope slide or are measured in a flow-througlh fluorimeter (FACS).

The expression “automated detection” encompasses methods which, entirely or only in constituent steps, replace the manual labour of human personnel and which are used, in particular, in steps of the detection procedure or in association with the subsequent documentation or information processing. This involves the steps of sample preparation, sample staining, sample reading and information processing.

The detection can be effected by means of absorption measurements, reflection measurements or fluorescence measurements.

The expression “diagnostic expert system” encompasses computer software which converts the image information into a proposed diagnosis. This expert system is able to consolidate all the information available, or parts thereof, into a proposed diagnosis on the basis of the parameters which are present in the software or of external information which the software can access. Should further parameters be required for substantiating the proposed diagnosis the software can propose the collection of these parameters or automatically request it by coupling to suitable analytical equipment within the sense of a reflex algorithm.

The expression “amplification system” encompasses biochemical methods which increase the signal intensities so as to produce a signal/noise ratio which is more favourable for specifically detecting a molecular marker. This is normally achieved by employing additional antibodies and/or enzymic detection reactions.

It is possible to use the present invention to specifically detect pathologically altered cells, e.g. carcinomas and their precursors, in the cervix. The invention also relates to a kit for implementing a method according to the invention, with this kit containing the following components:

(a) Reagents for detecting at least two of the molecular markers, namely labelled and/or unlabelled antibodies against her2/neu, p16. p53, MN, mdm-2, bcl-2, EGF receptor and HPV L1, and also labelled DNA probes which contain the regions of the HPV viral genome.

(b) Customary auxiliary substances, such as buffers, supports, signal amplification substances, staining reagents, etc.

(c) Automated methods for sample preparation and staining and for signal detection.

(d) Protocols and reagents for staining cell lines as a control reaction.

The above comments apply to the individual components of the kit. Individual components, or several components, of the kit can also be used in altered form.

It is possible to use the present invention to detect carcinomas and their precursors in cervical smears either manually or using methods which are partly or fully automated. Since the results, which are achieved in accordance with the invention, from the simultaneous detection of at least two molecular markers do not undergo any subjective assessment but, on the contrary, promote objective, automated detection of pathological changes in biological materials, the morphological findings, for example those made in a PAP test, can be supplemented with objective parameters, for example in the form of reflex testing as well. Because the methods can be implemented rapidly, and this implementation can be automated, they are suitable for large-scale screening methods which are economical with regard to costs and personnel.

Consequently, the present invention represents an important contribution to the specific detection of tumour cells and their precursors in connection with the early recognition of cervical cancer in the diagnosis of smears.

### EXAMPLES

Protocols for implementing the described invention are given below by way of example. While precise reaction conditions are specified, in these examples, for the respective antibodies or DNA probes, various parameters, such as incubation temperature and washing temperature, incubation times and washing times, and the concentration of antibodies and other reagents, can be varied dependent on the respective antibodies or DNA probes. In the same way, amplification systems which are described here can be omitted or added in.

Le A 35 010

### Description of an Experiment for Using Specific Antibodies to Simultaneously Detect Two Molecular Markers in Cervical Smears

Preparation techniques such as Thin-Prep (from Cytyc) are used to apply the cells, which are stored in Preservcyt (From Cytyc), to microscope slides (MS). The cells are fixed for 3 minutes with cold ethanol and, after that, the microscope slide is washed in PBS (137 mM NaCl, 3 mM KCl, 4 mM Na₂HPO₄, 2 mM KH₂PO₄). After nonspecific binding sites have been blocked, at room temperature (RT) for 30 min, with 5% foetal calf serum in PBS buffer, the MS are simultaneously incubated, for 60 min in a moist chamber, with two specific monoclonal antibodies (her2/neu (clone 3B5, 20 μg/ml) and p16 (clone DCS 50.1, 10 μg/ml) from Oncogene Science/BAYER). One of the antibodies is coupled to fluorescein while the other is coupled to biotin. After each of the following incubation steps, the MS are in each case washed 3 times with PBS for 5 min. Two different staining systems are used, one after the other, for detecting the individual antigen-antibody bindings. For the first staining system, the MS are incubated for 30 min with streptavidin-Cy3 (Mobi Tec, 5 μg/ml in PBS containing 5% calf serum), and then, after that, with anti-streptavidin-biotin (Vector; 2 μg/ml in PBS containing 5% calf serum) for a further 30 min in order to amplify the signal. Finally, the MS are incubated once again with streptavidin-Cy3. The second staining system consists of the Alexa Fluor 488 Signal Amplification Kit™ (Molecular Probes, order No. A-11053). Finally, the MS are covered with Mowiol™ (Hoechst).

### Description of an Experiment for Using Specific Antibodies to Simultaneously Detect Three Molecular Markers in Cervical Smears

Preparation techniques such as Thin-Prep (from Cytyc) or Cyto-Spin (from Shandon) are used to apply the cells, which are stored, for example, in Preservcyt (from Cytyc), to microscope slides (MS). The cells are fixed for 3 minutes with cold ethanol and the microscope slides are then washed in PBS (137 mM NaCl, 3 mM KCl, 4 mM Na₂HPO₄, 2 mM KH₂PO₄, pH 7.4). After nonspecific binding sites have been blocked, at room temperature (RT) for 30 min, with 5% foetal calf serum containing 4 mmol of levamisole (from Sigma)/1 in PBS buffer, the MS are incubated simultaneously, at room temperature for 60 min and in a moist chamber, with three specific monoclonal antibodies (her2/neu (clone 3B5, 20 μg/ml), bcl-2 (clone 100, 2 μg/ml) and p16 (clone DCS 50.1. 10 μg/ml) from Oncogene Science/BAYER). The first antibody is coupled to fluorescein while the second is coupled to digoxigenin and the third to biotin. After each of the following incubation steps, the MS are in each case washed 3 times with PBS for 5 min. Three different staining systems are used one after the other for detecting the individual antigen-antibody bindings. For the first staining system, the MS are incubated for 30 min with streptavidin-Cy3 (Mobi Tec. 5 μg/ml in PBS containing 5% calf serum containing 4 mmol of levamisole/1), and, after that, with biotinylated anti-streptavidin antibody (Vector: 2 μg/ml in PBS containing 5% calf serum containing 4 mmol of Levamisole/1) for a further 30 min in order to amplify the signal. Finally, the MS are incubated once again with streptavidin-Cy3. The second staining system consists of the Alexa Fluor 488 Signal Amplification Kit™ (Molecular Probes, order No. A-11053). For the third staining system, the MS are incubated with a conjugate consisting of sheep anti-digoxigenin antibody and alkaline phosphatase (DAKO, diluted 1:150 in PBS containing 5% calf serum containing 4 mmol of Levamisole/1) for 30 min. The fluorescent read-out is carried out using the ELF97 Cytological Labelling Kit (order No. E 6602) from Molecular Probes in accordance with the manufacturer's instructions. Finally, the MS are covered with Mowiol™ (Hoechst).

### Description of an Experiment for Using a Specific Antibody and an HPV DNA Probe for Simultaneously Detecting Two Molecular Markers in Cervical Smears

When combining an immunocytochemical antibody staining with an in situ DNA hybridization, the sample preparation and fixing, and the blocking and the incubation with a primary antibody, are carried out as in the above-described protocol for antibody staining. The primary antibody is a fluorescein-coupled antibody directed against the protein p16 (clone DCS 50.1), supplied by Oncogene Science/BAYER, and is used at a concentration of 10 μg/ml. For detecting the antigen-antibody binding, the MS are first of all incubated, at room temperature (RT) for 30 min, with a secondary antibody. i.e. rabbit anti-FITC (MoBiTec), at a concentration of 15 μg/ml, and then with an alkaline phosphatase-coupled goat anti-rabbit antibody, supplied by Dianova, at a dilution of 1:100 in PBS. For the staining system, the MS are incubated, at room temperature for 5 min, with the chromogenic alkaline phosphatase substrate Fuchsin, supplied by DAKO, in accordance with the manufacturer's instructions. For fixing the stain, the MS are subsequently fixed with 5% paraformaldehyde in PBS for 5 min. The insitu staining then follows. For this, the MS are first of all washed twice in 2×SSC (0.3M NaCl, 30 mM Na citrate) and then digested, at 37° C. for 60 min. with 40 μg of RNase/ml in 2×SSC. After 2 two-minute washing steps with PBS, digestion is carried out, at 37° C. for 10 min, with 2 μg of proteinase K/ml. After 3×2 min washes with distilled water, 20 μl of ready-to-use digoxigenin-labelled HPV16 DNA probe (Kreatech) in hybridization buffer (Kreatech) are added to the MS, which are then covered with cover slips, sealed, heated at 94° C. for 8 min for denaturation, and then incubated at 37° C. overnight. After the hybridization solution has been removed, the MS are once again blocked, for 30 min, with 3% rabbit serum in Boehringer blocking buffer (0.05 g of blocker (Boehringer 1201107) in 5 ml of blocking buffer (150 mM NaCl, 100 mM tris-HCl, pH 7.5)). For the chromogenic read-out, the MS are incubated consecutively, in each case at RT for 15 min, with DAKO peroxidase-coupled rabbit anti-digoxigenin antibody (diluted 1:150 with blocking buffer), Dig-Tyramide (5.71 μg/ml) (prepared as described in: Hopman et al., 1998, J Histochem Cytochem., 46(6), 771-777) in PBS/0.1M imidazole, pH 7.6/0.001% H₂O₂ and, once again, with peroxidase-coupled rabbit anti-digoxigenin antibody (DAKO, diluted 1:150 in TBST (50 mM tris/HCl, 0.3M NaCl, 1% Tween 20, pH 7.6) containing 3% rabbit serum). In between, the MS are in each case washed 3×5 min with PBST. For the chromogenic color reaction, the MS are stained for 10 min with DAB substrate (from DAKO) in accordance with the manufacturer's instructions. Finally, the MS are covered in Mowiol™.

### Description of an Experiment for Automatically Detecting Abnormal Cells which have been Stained by the Simultaneous Detection of at Least Two Molecular Markers

Cervical smears, in which at least two molecular markers have been detected with antibodies using the above-described methods, are interpreted using a fluorescence microscope having an actuatable cross-stage for up to 8 microscope slides (Olympus AX70 with Multicontrol-Box 2000-3 and analySIS “Modul Stage™” drive software), and a modified version of the analySIS 3.0 software from Soft Imaging Systems GmbH (SIS), which, as the “Grabbit Dual Pro” SIS package, contains additional modules, in particular an FFT (=“Fast Fourier Transformation”) module, an MIA (=“Multiple Image Alignment”) module and a C module. High-resolution black-white (MV2 Slow scan Camera, 12 bit, from SIS) and color (DXC-950P 3CCD chip from Sony) cameras are used for the image recordings. In combination, these systems are suitable for a 16-bit Grauton image analysis and for color image analysis in the RGB and HSI color spaces up to a 24-bit image depth.

After calibrating the actuatable cross-stage (analySIS “Module Stage”), the positions of in all up to 8 microscope slides are recorded, relative to the “logical zero point”, by defining eight consecutive stage paths in the “automation” menu (analySIS “Module Grains”). The total area of the biological samples, which the automated Thin-Prep (Cytyc) methodology ensures are always located in a defined re(,ion of the microscope slides is broken down, in a comprehensive manner, into individual regions which are of 571.2 μm×457.96 μm in size and which correspond to the image sector of a photograph at 20-told magnification using the microscopic unit. Each biological sample is analyzed through one stage path. Each stage path consists of 750 individual stage path positions (30 lines, 25 columns) which the microscope describes horizontally, in a meandering manner, as a series of stage path positions, such that each region of the biological sample of the given microscope slide position is detected in an abutting manner, and not in an overlapping manner, by means of a stage path position. Consequently, an identical stage path is in each case defined for each preparation on the working stage. Adding the 8 stage paths in the “automation” menu makes it possible to analyze all the preparations which are positioned on the avoiding stage in an automated manner.

In general, after having been excited with the 3 different fluorescence wavelengths, each stage path position is in each case photographed 1×using the high-resolution B/W camera and appropriate fluorescence color liters. The fluoresence intensities of the individual photographs are then measured. This is done using the “measurement” recording card in the “automation” menu. Color mixtures, which correlate with the fluorescence intensities of the individual images at identical stage path positions, are produced by superposing individual blue, red and green images. There then follows the “phase analysis” that is the regions of given color mixtures are measured and reproduced quantitatively as areas. In this connection, given color mixtures are characteristic of pathologically altered cells, which means that it is possible to make a diagnostic prediction.

In detail, for the fluorescence measurements using several molecular markers, a black and white photograph is made, per stage path position, for each fluorescence filter channel. Colors are then assigned to the individual images belonging to a stage path position and the resulting false color images are superimposed, using the “FIP module”, such that color mixtures are obtained, dependent on the fluorescence intensities, by “adding” the different fluorescence colors. For example, a yellow secondary color is obtained it the corresponding image position when there is simultaneous fluorescence coloration of equal strength in the red and green color channels. Depending on the strength of the fluorescence intensities, the secondary colors can also tend towards the red or the green. In a similar way, a white secondary color is formed when blue, green and red fluorescences of equal intensity are superimposed. These superimposed images are then measured by setting a threshold value and subsequently performing a “phase analysis”, with this taking place directly by setting a color threshold in the RGB mode (images menu). All these steps can be performed manually, but they can also be carried out in an automated manner by creating macros in the “Extras” menu under “plot macros” and subsequently retrieving them in the “automation” menu. Finally, a quantitative assessment for particular color mixtures is obtained in the form of an area value (=area on the biological sample which possesses a precisely defined secondary color due to having been stained with at least 2 molecular markers at a particular intensity). Both the analyzed images and the area values for the respective stage path positions can be stored automatically using the “protocol” recording card in the “automation” menu. In this way, a quantitative assessment with regard to the fluorescence intensities or color mixtures measured in this region is obtained for each stage path position. The values of the color mixtures at the individual stage path positions (=superimposition of three individual photographs) are displayed for each stage path (=individual preparation) in one Excel data file in each case. The Excel tables consequently consist of 750 lines, for the 750 individual stage path positions per stage path, and of 8 columns for the 8 different color mixtures. Since particular color mixtures (e.g. yellow color mixtures when displaying two individual markers in red and green) can be attributed to several disease-associated markers being expressed at a simultaneously high level, these stage path positions are labelled (=“flagging”) as being “pathological”, such that the corresponding fluorescent photographs can subsequently be examined visually, for control purposes, as individual images. The areas given in an Excel table for the eight color mixtures are accredited using a macro in Excel, i.e. all the values in a column (=a particular color mixture) are summed. Consequently, a numerical value for each of the eight secondary colors is obtained for a given biological preparation (=stage path). If the sum of particular color mixtures which are characteristic of pathologically altered cells (such as the yellow color mixture which has already been mentioned above) exceeds a critical minimum value, the biological sample, or the given cervical smear, which is analyzed by the corresponding stage path is classified as being pathological. Consequently, a diagnostic prediction with regard to the biological sample is automatically achieved by quantitatively analyzing the accredited individual fluoresence intensities.

### Description of an Experiment Using Specific Antibodies for Simultaneously Detecting Two Molecular Markers in a Flow-through Cytometer

Smear cells which have been stored in Preservcyt ((Cytyc) are firstly pressed through a 100 μm pore-size nylon cloth in order to break cell clumps up into individual cells. After the cells have been sedimented by centrifugation, they are washed 1× with PBS and subsequently permeabilized using one of the following methods: a) OPF method (ORTHOPermeaFix™. b) F&P (FIX&PERM Cell Permeablization Kit, Imtec) and c) MWH (microwave heating). All of the methods have to be carried out either in accordance with the manufacturer's instructions or in accordance with Millard et al. (Clin Chem 1998, 44, 2320-2330). The permeabilized cells are incubated for 60 min with the two mouse antibodies (her2/neu (clone 3B5, 20 μg/ml) and p16 (clone DCS 50.1, 10 μg/ml), supplied by Oncogene Science/BAYER). The first antibody is coupled to fluorescein while the second is coupled to biotin. After each of the subsequent incubation steps, the cells are sedimented and washed a total of 2 times with PBS. Two different staining systems are used consecutively for detecting, the individual antigen-antibody bindings. In the case of the first staining system, the cells are incubated for 30 min with streptavidin-Cy3 (Mobi Tec. 5 μg/ml). The second staining system consists of the Alexa Fluor 488 Signal-Amplification Kit™ (Molecular Probes, order No. A-11053). The cells are subsequently taken up in ISOTON II (Coulter) and measured in a flow-through cytometer (FACScan, Becton Dickinson), which is fitted with laser excitation for green and red fluorescence. The fluorescence intensities can be depicted graphically. The relative and absolute number of the negative cells, and of the singly positive or doubly positive cells, can be detected after defining threshold values for the signal intensities. A diagnostic expert system converts this information into a proposed diagnosis.

1. Method for detecting tumour cells and their precursors in uterine cervical smears by simultaneously detecting at least two molecular markers in a cell.

2. Method according to claim 1, characterized in that the markers are selected from at least one of the following groups: tumour suppressor genes, apoptosis genes, proliferation genes, repair genes and viral genes.

3. Method according to claims 1 and 2, characterized in that at least one of the following markers is present in the combination: her2/neu, p16, p53, MN, mdm-2, bcl-2. EGF receptor, and specific DNA from the HPV subtypes 6, 11, 16, 18, 30, 31, 33, 35, 45, 51 and 52.

4. Method according to one of claims 1-3, characterized in that the marker combinations her2/neu with p16 or EGF-R with p16 or p53 with her2/neu or her2/neu with mdm-2 or bcl-2 with p16 or bcl-2 with bcl-2/neu or p16 with p53 are present.

5. Method according to one of claims 1-4, characterized in that 3 markers are detected.

6. Kit for implementing the method according to one of claims 1-5.

7. Kit according to claim 6, characterized in that the reagents are antibodies or nucleic acids.

8. Kit according to claims 6 and 7, characterized in that the antibodies or nucleic acid probes are read directly or indirectly using fluorescent or chromogenic dye substances.

9. Method according to one of claims 1-8, characterized in that it enables abnormal cells to be detected in an automated manner, characterized in that at least two markers are detected and the signal intensities fire combined and summated.

10. Method according to claim 9, characterized in that the automatic information processing is combined with a diagnostic expert system which enables the image information to be consolidated into a proposed diagnosis and, where appropriate, enables reflex testing to be carried out.

11. Entire process according to claims 1-10, characterized in that the process consists of fully automatic sample preparation, sample staining, sample reading and information processing or subprocesses which comprise at least two of the given subprocesses.
